(19) United States
(12) Patent Application Publication
(10) Pub. No.: US 2013/0158621 A1
(43) Pub. Date: Jun. 20, 2013

(54) ECTOPIC-TRIGGERED PARA-HIS STIMULATION

(71) Applicants: Jiang Ding, Shoreview, MN (US); Allan C. Shuros, St. Paul, MN (US); Dan Li, Shoreview, MN (US); Yinghong Yu, Shoreview, MN (US); Stephen J. Hahn, Shoreview, MN (US)

(72) Inventors: Jiang Ding, Shoreview, MN (US); Allan C. Shuros, St. Paul, MN (US); Dan Li, Shoreview, MN (US); Yinghong Yu, Shoreview, MN (US); Stephen J. Hahn, Shoreview, MN (US)

(21) Appl. No.: 13/716,810

(22) Filed: Dec. 17, 2012

Related U.S. Application Data
(60) Provisional application No. 61/578,056, filed on Dec. 20, 2011.

(51) Int. Cl. A61N 1/365

(52) U.S. Cl. CPC A61N 1/36592
USPC 607/17

(57) ABSTRACT

Ectopic cardiac activity can be detected, such as in the absence of a diagnosed tachyarrhythmia episode. In response to the detected ectopic activity, electrostimulation can be provided to a para-Hisian region, such as to activate natural cardiac contraction mechanisms or to interrupt re-entrant cardiac activity. Subsequent ectopic cardiac activity can be detected, and subsequent electrostimulation can be provided to the para-Hisian region, such as according to one or more adjustable electrostimulation parameters.

### CLAIM OF PRIORITY

This application claims the benefit of priority under 35 U.S.C. §119(e) of Ding et al., U.S. Provisional Patent Application Ser. No. 61/578,056, entitled “ECTOPIC-TRIGGERED PARA-HIS STIMULATION”, filed on Dec. 20, 2011, which is herein incorporated by reference in its entirety.

### BACKGROUND

A natural cardiac activation sequence can include an electrical impulse that can originate at a sinoatrial node (SA node), pass through intermodal atrial pathways, and arrive at an atrioventricular node (AV node). From the AV node, a His bundle and its various branches can be activated, and electrical signals can ultimately reach an apex of the myocardium using a heart's Purkinje system. In some patients, the natural cardiac activation sequence can be disturbed. For example, abnormal activation of myocytes, or individual cardiac muscle cells or groups of muscle cells, can cause abnormal fluctuations in heart rate.

Intrinsic electrical stimuli of the heart, such as in the presence of various myocardial substrate modifications (e.g., infracted or non-conducting areas), can re-enter an original activation circuit and trigger a new activation. Such re-entrant circuits can lead to elevated heart rates that can be fatal. For example, tachycardia episodes, as a type of persistent elevated heart rate, can occur.

Ectopic cardiac activity, such as due to an abnormal activation of myocytes, can be problematic for some patients. For example, ectopic cardiac activity can include an ectopic event, or ectopic beat, such as a cardiac activation that can be caused by a signal other than a naturally-occurring impulse originating at the SA node. Ectopic cardiac activity can lead to re-entrant circuitry, such as can cause an elevated heart rate or other arrhythmia. There can be various ectopic foci of activation of cardiac tissue, such as in ventricular or atrial tissues. Various methods for detecting ectopic events have been proposed. For example, Ding et al., U.S. Pat. No. 6,453,192, entitled DETECTION OF VENTRICULAR ECTOPIC BEATS USING VENTRICULAR ELECTROGRAM, refers to determining an ectopic beat using a time interval between an earliest activation of a ventricular polarization and a peak of a following ventricular depolarization.

Medical devices, such as implantable medical devices, can be used to perform one or more tasks including monitoring, detecting, or sensing physiological information in or otherwise associated with the body, diagnosing a physiological condition or disease, treating or providing a therapy for a physiological condition or disease, or restoring or otherwise altering the function of an organ or a tissue. Examples of an implantable medical device can include a cardiac rhythm management device, such as a pacemaker, a cardiac resynchronization therapy device, a cardioverter or defibrillator, a neurological stimulator, a neuromuscular stimulator, or a drug delivery system.

In an example, cardiac rhythm or function management devices can sense heart contractions or deliver electrostimulation to evoke responsive heart contractions. In an example, one or more of these functions can help improve a patient's heart rhythm or can help coordinate a spatial nature of a heart contraction, either of which can improve cardiac output of blood to help meet a patient's metabolic need.

Some cardiac rhythm or function management devices can be configured to deliver energy at or near the His bundle to achieve pacing via natural conduction pathways, such as via Purkinje fiber conduction of electrical impulses. Various devices for delivering signals to an electrode near a His bundle have been proposed. For example, Zhu et al., PCT Patent Publication No. WO 2010/071849, entitled DEVICES, METHODS, AND SYSTEMS INCLUDING CARDIAC PACING, refers to delivering an anti-tachyarrhythmia pacing pulse to an electrode near a His bundle in a right ventricle of a heart.

### Overview

The efficiency and efficacy of a cardiac response to artificial electrostimulation can depend on many factors, such as including how and where an electrostimulation can be provided. An efficient electrostimulation delivery technique can include pacing at the His bundle. Electrostimulation delivered to the His bundle can activate a heart's natural conduction mechanisms, such as the left and right branch bundles and Purkinje fibers, to produce an efficient and coordinated cardiac response.

This document describes, among other things, systems, methods, machine-readable media, or other techniques that can involve using a medical device to detect a first premature ventricular contraction (PVC) of a heart, or ectopic heart beat, such as in the absence of a diagnosed tachyarrhythmia episode. In response to detecting the first PVC, an electrostimulation can be delivered to a location at or near a His bundle (e.g., a para-Hisian region). Subsequent premature ventricular contractions can be detected, and in response, one or more subsequent electrostimulations can be delivered. Various electrostimulation parameters can be adjusted, such as including timing, waveform, or duration parameters, or how many electrostimulations are delivered in response to a detected PVC.

The present inventors have recognized, among other things, that a problem to be solved can include treating ectopic cardiac events, such as in the absence of a diagnosed tachyarrhythmia episode. The present inventors have recognized, among other things, that a problem to be solved can include treating ectopic cardiac events using systems and methods that are tolerable to a patient (e.g., using electrostimulation therapies at low amplitudes such that the electrostimulation does not cause patient discomfort), and promote longevity of the devices used to deliver the treatments (e.g., battery-powered, implantable devices). In an example, the present subject matter can provide a solution to these and other problems, such as by detecting ectopic cardiac activity or providing electrostimulation to a para-Hisian region of a heart. In an example, providing electrostimulation to a para-Hisian region can disrupt re-entrant activity or reduce the likelihood of forming a re-entrant circuit.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates generally an example of a dysfunctional portion of myocardial tissue.

FIG. 1B illustrates generally an example of impulse propagation along a re-entry loop.

FIG. 1C illustrates generally an example of EKG waveforms exhibiting premature cardiac activity.

FIG. 2 illustrates generally an example of a system that can include an electrical energy delivery circuit, a detector circuit, or a processor circuit.

FIG. 3 illustrates generally an example of an ambulatory or implantable medical device (IMD) in a subject.

FIG. 4 illustrates generally an example of a system configurable to deliver electrostimulation energy to a subject.

FIG. 5 illustrates generally an example of a system that can include an IMD coupled to electrode leads disposed in or near a heart.

FIG. 6 illustrates generally an atrial lead.

FIG. 7A illustrates generally an example that can include disrupting a re-entry loop in cardiac tissue.

FIG. 7B illustrates generally an example of a coordinated response of myocardial tissue.

FIG. 8 illustrates generally an example that can include providing electrostimulation to a heart.

FIG. 9 illustrates generally an example that can include identifying an ectopic event and providing electrostimulation using an origin-dependent parameter.

FIG. 10 illustrates generally an example that can include determining a His bundle capture status.

FIG. 11 illustrates generally an example that can include adjusting an electrostimulation parameter.

FIG. 12 illustrates generally an example of a waveform indicative of cardiac electrical activity of the heart.

FIG. 13 illustrates generally an example that can include providing a first electrostimulation to a para-Hisian region, adjusting an electrostimulation parameter, and providing a second electrostimulation to the para-Hisian region.

FIG. 14A illustrates generally an example that can include increasing a number of electrostimulations.

FIG. 14B illustrates generally an example that can include adjusting a para-Hisian electrostimulation delay.

FIG. 14C illustrates generally an example 1402 that can include adjusting an electrostimulation parameter when ectopic events are detected at different origins or different rates.

FIG. 15 illustrates generally a chart that can include information about an electrostimulation trend.

### DETAILED DESCRIPTION

A conduction pathway of the heart that can be used to activate cardiac contractions can originate in a sinoatrial (SA) node in a right atrium of the heart. Intrinsic electrical impulses, such as generated at the SA node, can trigger atria of the heart to contract. From the SA node, a conduction pathway can lead electrical impulses to an atrioventricular (AV) node, located between the atrium and the ventricle. Following a delay at the AV node, conduction can continue through the His bundle to the left and right bundle branches, then to the Purkinje fibers and the apex of the heart, and finally up and around to the ventricular myocardium to produce a coordinated cardiac contraction, such as of both the left and right ventricles.

Cardiac contractions initiated using a natural conduction pathway, such as intrinsic contractions, can be faster and more efficient than paced contractions, such as contractions initiated using apical or biventricular pacing. Accordingly, providing stimulation energy (e.g., a pacing energy or electrostimulation) to a portion of the natural conduction pathway, such as the His bundle, can activate the faster-conducting fibers, such as the Purkinje fibers, to enable an efficient physiological stimulation and, in some examples, improved hemodynamics as compared to apical or biventricular paced activations.

In an example, some cardiac activity can originate from locations other than the SA node. For example, an ectopic heart beat can occur. An ectopic heart beat can be caused by a dysfunctional conduction pathway in myocardial tissue, such as in atrial or ventricular myocardial tissue. Ectopic heart beats can be irregular beats that can occur suddenly or rapidly, such as before a previous cardiac cycle ends. Various systems and methods for detecting ectopic beats have been proposed. Ding et al., in U.S. Pat. No. 6,453,192 entitled DETECTION OF VENTRICULAR ECTOPIC BEATS USING VENTRICULAR ELECTROGRAM, which is hereby incorporated herein by reference in its entirety, describes using interval information to identify ectopic beats and identify an origin of ectopic excitation.

In an example that can include properly functioning myocardial conduction pathways, myocardial tissue can transmit impulses substantially omnidirectionally. In an example, the properly functioning pathways can use rapid myocardial or other conduction pathways, and the cells of the myocardial tissue can transmit impulses about one time over a particular activation interval. In an example, a dysfunctional myocardial conduction pathway can be slowed or blocked relative to other myocardial conduction pathways, such as due to disease or tissue damage, among other reasons.

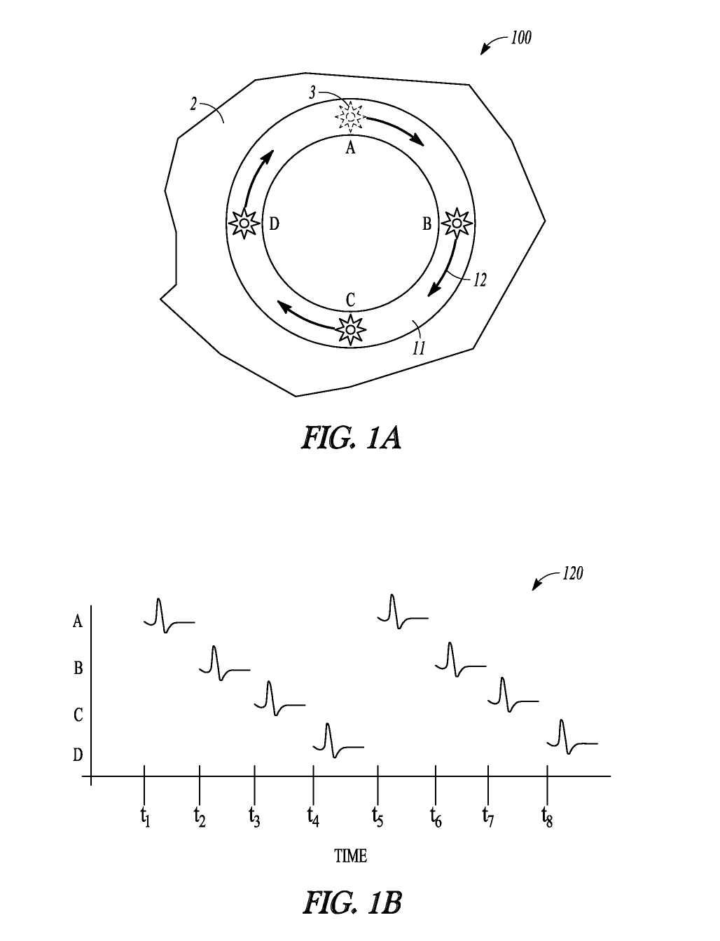

FIG. 1A illustrates generally an example 100 of a portion of myocardial tissue 2. In an example that can include a dysfunctional myocardial conduction pathway, such as including a partial or complete blockage of all or a portion of a conduction pathway, a re-entrant loop 11 can form in the myocardial tissue 2. For example, the re-entrant loop 11 can form as a result of a late-arriving impulse. In an example, a late-arriving impulse can arrive outside of an initial activation interval of the cells of the myocardial tissue 2 (e.g., the impulse can arrive after a refractory period expires). In an example, the re-entrant loop 11 can form when the cells in the myocardial tissue 2 respond to a first impulse, and then respond again to a subsequent related impulse, for example after an impulse has traveled in a loop along a conduction pathway (e.g., a dysfunctional conduction pathway). The re-entrant loop 11 can include an impulse that can travel in a loop, such as instead of or in addition to traveling along the myocardium to produce a coordinated cardiac contraction. The example re-entrant loop 11 of FIG. 1A illustrates a hypothetical circular loop; however, physiological re-entrant loops can occur along any pathway shape such that a beginning point (e.g., an impulse origination site) and end point (e.g., an impulse reception site) can generally coincide.

In the example of FIG. 1A, the myocardial tissue 2 can be excited and an impulse 3 can originate at a location A. The impulse 3 can travel along the myocardium, such as in a first direction 12. The impulse 3 can propagate through the myocardium, such as along the first direction 12 from location A, to location B, to location C, and to location D. In an example, the myocardial tissue 2 can be dysfunctional, and the impulse 3 can propagate from location D back to location A, such as after expiration of a refractory period of the myocardial tissue 2 cells at location A. In an example, non-homogeneity of a refractory period associated with respective portions of the myocardial tissue 2 (e.g., the portions of the tissue at locations A, B, C, or D, among others) can cause re-entrant behavior.

FIG. 1B illustrates generally an example of a chart 120 that can illustrate propagation of an impulse along a re-entry loop. For example, at time t₁, an impulse can arrive at the myocardial tissue 2 at a location A. At t₂, the impulse can arrive at a location B, at t₃ the impulse can arrive at a location C, and at t₄ the impulse can arrive at a location D. In an example, the cells at location A can be reactivated after expiration of a refractory period, such as in response to the impulse at the location D. At t₅, the impulse can reactivate the cells at location A, and a re-entry loop can form.

In an example, after the re-entrant loop 11 forms, the loop can continue to transmit impulses, such as at a rate that can exceed a normal sinus rate. Sustained abnormal cardiac rhythms can result, and some patients can experience atrial flutter or tachycardia. In an example, re-entrant cardiac activity can cause ectopy, or a premature contraction event, such as a premature ventricular contraction (PVC) event or a premature atrial contraction (PAC) event.

FIG. 1C illustrates generally an example of a chart 140 that can include multiple waveforms, such as EKG waveforms. In an example, a first waveform 141 can indicate a normal sinus rhythm, such as without premature contraction activity. In an example, a second waveform 142 can indicate ectopy, such as including a premature ventricular contraction (PVC) 152. In an example, the PVC 152 can include a ventricular contraction that can occur before an anticipated sinus ventricular contraction (e.g., before an interval At elapses, such as an interval between adjacent R-waves in a normal sinus rhythm). In an example, a non-premature contraction 154 can occur adjacently sequentially preceding the PVC 152. That is, an otherwise normal sinus rhythm can be interrupted by a premature contraction event, such as a PVC. In an example, a third waveform 143 can indicate ectopy, such as including a premature atrial contraction (PAC) 153.

In an example, ventricular ectopy, such as due to re-entrant cardiac activity, can progress to other ventricular dysfunction, such as including ventricular fibrillation. However, a re-entrant loop, such as the re-entrant loop 11, can be dissolved, such as using the systems and methods described below.

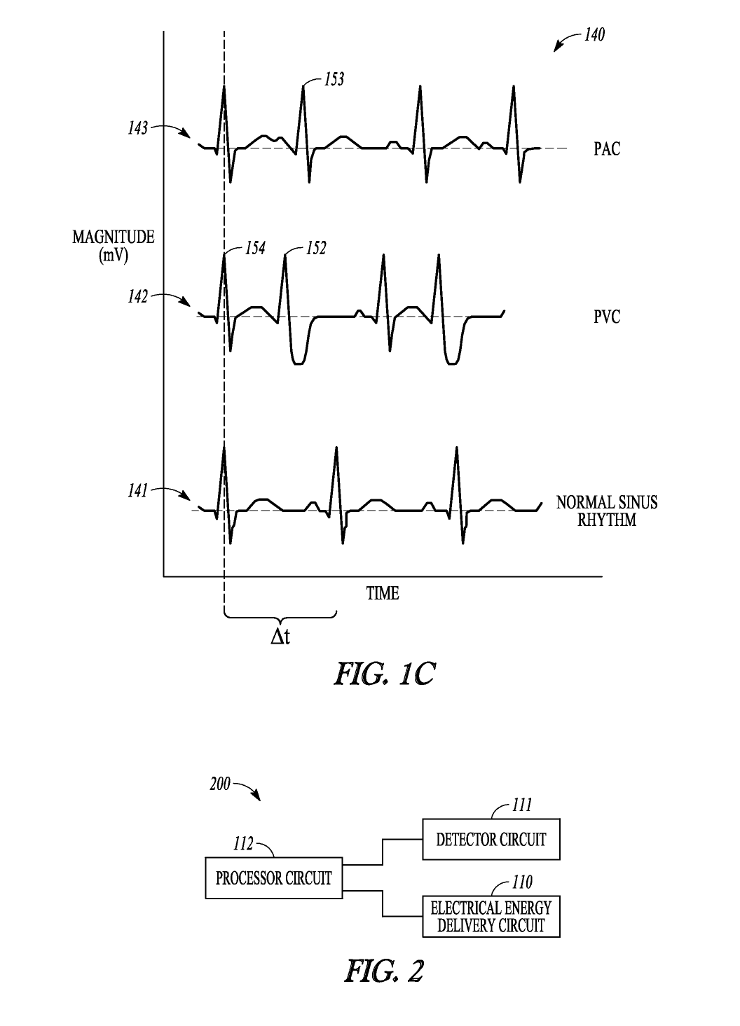

FIG. 2 illustrates generally an example of a system 200 that can include an electrical energy delivery circuit 110, a detector circuit 111, and a processor circuit 112. In an example, the electrical energy delivery circuit 110 can be configured to generate or provide an electrostimulation, such as can be delivered to a subject body using an implantable or external lead to evoke a cardiac response such as a cardiac depolarization. In an example, the electrostimulation can be configured to stimulate a His bundle. His bundle stimulation can be provided, for example, using an electrode, such as coupled to the electrical energy delivery circuit 110. The electrode can be disposed in a right ventricle, such as at one or more locations along an interventricular septum, a right ventricular outflow tract septum, a right atrium, or one or more other locations near the His bundle.

In an example, the detector circuit 111 can be configured to receive information about a heart, for example, over at least a portion of a cardiac cycle. In an example, the detector circuit 111 can be configured to receive information such as including one or more of: electrogram or electrical cardiogram (EKG) information (e.g., an evoked response EKG, a subcutaneous EKG, or other electrical activity information); heart sound information, such as can be received from a heart sound sensor such as a microphone; acceleration information, such as can be received from an accelerometer configured to provide an indication of mechanical cardiac activity; pressure information, such as can be received from a pressure sensor configured to provide an indication of a pressure, such as a central venous pressure (CVP); thoracic or other impedance information; or other information indicative of cardiac activity.

The processor circuit 112 can be configured to determine a characteristic of the received cardiac activity information, such as over at least a portion of a cardiac cycle. In an example, a characteristic can include, among others, at least one of a width, amplitude, polarity, slope, or latency of a QRS complex, an R-wave timing, a pressure, an indication of mechanical motion provided by an accelerometer, or an impedance.

In an example, one or more other characteristics can be used, such as a measure of contractility, synchrony, or cardiac output, among others. Information from the determined characteristics can be used to determine, among other things, a time interval, such as can be used to identify an ectopic heart beat.

In an example, the processor circuit 112 can be configured to detect premature ventricular activity, such as a PVC, such as in the absence of a diagnosed tachyarrhythmia episode. For example, the processor circuit 112 can be configured to identify a single, individual, or particular PVC or a single, individual, or particular ectopic event, such as in an otherwise normal sinus rhythm. In response to detecting the PVC or ectopic event, the processor circuit 112 can initiate an electrostimulation, such as using the electrical energy delivery circuit 110. For example, in response to the PVC or ectopic event, the processor circuit can initiate delivery of an electrostimulation to a subject, such as can be provided to a para-Hisian region of a subject's heart.

In an example, the processor circuit 112 can determine a cardiac stimulation diagnostic indication, such as using information received from the detector circuit 111. In an example, the cardiac stimulation diagnostic indication can be used to discriminate between His bundle cardiac capture via Purkinje fiber conduction, cell-to-cell conduction cardiac capture, and intrinsic conduction cardiac contraction, such as to provide an indication of an efficacy of para-Hisian electrostimulation. In an example, the processor circuit 112 can be configured to report (or make available) one or more cardiac stimulation diagnostic indications to an external module (e.g., an external programmer, directly to a clinician's handheld mobile device, email, etc.). In an example, the processor circuit 112 can be configured to provide a cardiac stimulation diagnostic indication for a plurality of cardiac cycles, count or store one or more of the results from the classification, such as in a histogram. In an example, when the His bundle capture percentage falls below a threshold, the processor circuit 112 can be configured to, among other things, provide an alert to an external module, reduce a stimulation energy to save power, increase a stimulation energy (e.g., the pacing threshold) to ensure His bundle capture, switch to a different pacing configuration (e.g., different pacing waveform, site, etc.), or initiate a test to determine the His bundle threshold. In an example, a success rate of His bundle capture or other cardiac capture can be trended, and the trended information can be provided to an external module and displayed to a user.

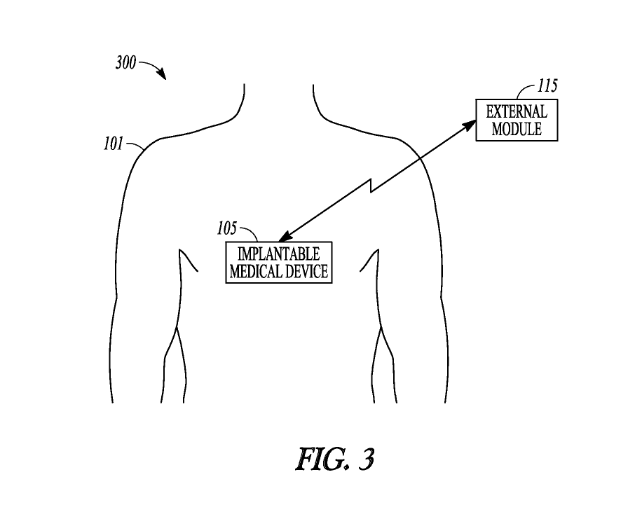

FIG. 3 illustrates generally an example of a system 300, such as can include an ambulatory or implantable medical device (IMD) 105 in a subject 101. The IMD 105 can be coupled, such as wirelessly, to an external module 115. In an example, the IMD 105 can include one or more of the electrical energy delivery circuit 110, the detector circuit 111, or the processor circuit 112. In an example, a portion of the functionality of one or more of the electrical energy delivery circuit 110, the detector circuit 111, or the processor circuit 112, can occur in the IMD 105, and another portion elsewhere (e.g., in an external component, such as a 12-lead EKG detector).

In an example, the IMD 105 can include a pacemaker, a defibrillator, or one or more other implantable medical devices. In an example, the IMD 105 can include an antenna configured to provide radio-frequency or other communication between the IMD 105 and the external module 115, or other external device. In an example, the external module 115 can include an antenna. In an example, the external module 115 can include a local medical device programmer or other local external module, such as within wireless communication range of the IMD 105 antenna.

In an example, the external module 115 can include a remote medical device programmer or one or more other remote external modules (e.g., outside of wireless communication range of the IMD 105 antenna, but coupled to the IMD 105, such as using a local external device, such as a repeater or network access point). In an example, the external module 115 can be configured to send information to or receive information from the IMD 105. The information can include medical device programming information, subject data, device data, or other instructions, alerts, or other information. In an example, the external module 115 can be configured to display information (e.g., information received from the IMD 105) to a user. Further, the local programmer or the remote programmer can be configured to communicate the sent or received information to a user or physician, such as by sending an alert (e.g., via email) of the status of the subject 101 or the system 300 components.

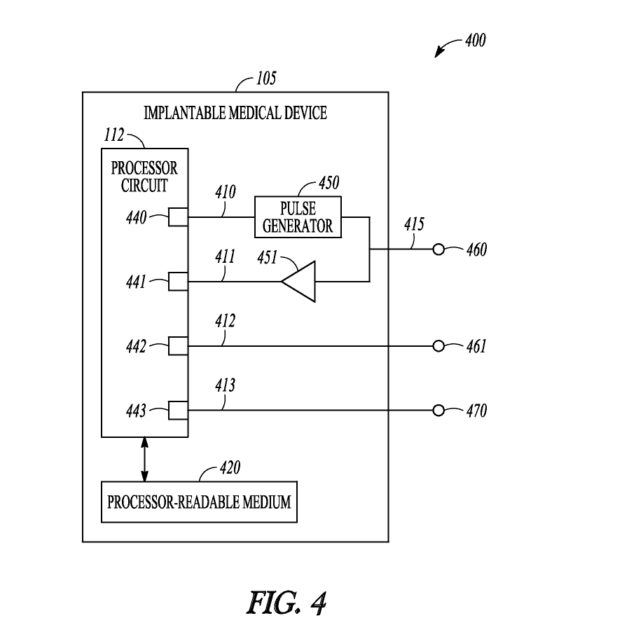

FIG. 4 illustrates generally an example of a system 400 that can be configured to deliver electrostimulation energy to a subject or sense a subject response. In an example, the system 400 can include the IMD 105, such as including systems configured to provide or deliver electrostimulation energy to a subject, or receive physiological activity information about subject.

In an example, the system 400 can include multiple electrostimulation or sense channels, such as a His bundle electrostimulation channel 410, a right ventricular sense channel 411, a right ventricular pace channel 412, or a sensor sense channel 413. In an example, the system 400 can include the processor circuit 112, and a processor-readable medium 420, such as can be accessed using the processor circuit 112.

In an example, the processor circuit 112 can include a plurality of data inputs and outputs. For example, a first data output 440 can be coupled to the His bundle electrostimulation channel 410, such as can be configured to provide control information to a pulse generator 450. The pulse generator 450 can be coupled to an electrode 460, such as can be disposed on a lead 415 or elsewhere. In an example, a first data input 441 can be coupled to the right ventricular sense channel 411, such as can be configured to receive, via a sense amplifier 451, an electrical signal from the electrode 460. In an example, the electrode 460 can be configured to be located in a right ventricle, such as in the septal region proximal to the His bundle, the right ventricular outflow tract, the free wall region, or another region of the right ventricle.

In an example, a second data output 442 can be coupled to the right ventricular pace channel 412, such as can be coupled to an electrode 461. A second data input 443 can be coupled to a first sensor 470, such as a pressure sensor configured to be disposed in a thoracic vena cava, such as to measure central venous pressure to provide an indication of a right atrial pressure. In an example, the processor circuit 112 can receive pressure information via an electrical signal and can interpret the pressure signal, such as using instructions provided on the processor-readable medium 420.

In an example, the system 400 can include additional pace or sense channels, such as an atrial pace channel, an internal thoracic pace/sense channel, such as can be configured to couple the processor circuit 112 to an internal thoracic location external to the heart (e.g., through one or more leads, electrodes, pulse generators, or sense amplifiers), or one or more other atrial or ventricular pace or sense channels, among others. The system 400 can include additional sense channels, such as can be configured to receive information from sensors such as accelerometers, pressure sensors, or electrodes, such as can be configured to measure electric field information. In an example, the IMD 105 can include one or more other right or left ventricular sense or pace channels, such as a right ventricular apex backup pacing channel.

In the example of FIG. 4, the processor circuit 112 can be an implantable component, an external component, or a combination or permutation of an implantable processor and an external processor. In an example, if at least a portion of the processor circuit 112 includes an external processor, then the processor circuit 112 can be configured to be communicatively coupled (such as via telemetry, RF, or other communication protocol) with the remaining components, such as the sense amplifier 451, the pulse generator 450, or the processor-readable medium 420. In an example, the implantable processor can be configured to have reduced or minimal functionality or power consumption. In an example, the processor circuit 112 can include an external processor for computing complex operations or to store large amounts of information. In an example, the processor circuit 112 can include a microcontroller, a microprocessor, a logic circuit, or other processor. In an example, the electrical energy delivery circuit 110 can include the pulse generator 450, and the detector circuit 111 can include the sense amplifier 451.

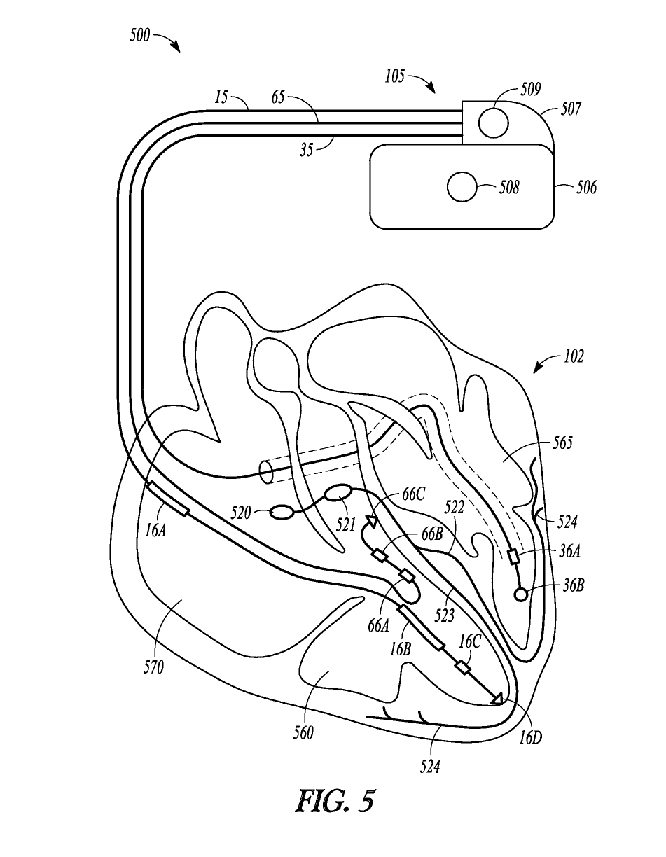

FIG. 5 illustrates generally an example of a system 500 that can include an IMD 105, a right ventricular apex lead 15, a left ventricular lead 35, and a right ventricular septum lead 65. The IMD 105 can include a housing 506 (or “can”) and a header 507. In an example, at least a portion of the exterior of the housing 506 or the header 507 can include an electrode, such as a housing can electrode 508, or a header electrode 509.

In an example, the right ventricular apex lead 15 can include a first electrode 16A that can be configured to be located in the superior vena cava of a heart 102, and a second electrode 16B, a third electrode 16C, and a fourth electrode 16D configured to be located in the right ventricle 560 of the heart 102. In an example, the first electrode 16A can include a proximal defibrillation coil electrode, or the second electrode 16B can include a distal defibrillation coil electrode, such as can be configured to deliver a high energy shock (e.g., 0.1 Joule or greater) to a heart 102.

In an example, the left ventricular lead 35 can include a fifth electrode 36A and a sensor 36B configured to be located in, on, or near the left ventricle 565 of the heart 102, such as within the coronary vasculature. In an example, the sensor 36B can include a distal pacing or sensing electrode, or a pressure sensor. The right ventricular septum lead 65 can include a sixth electrode 66A, a seventh electrode 66B, and an eighth electrode 66C, such as configured to be located along the septum in the right ventricle 560 of the heart 102. In an example, the right ventricular septum lead 65 can be configured to provide His bundle pacing along the septum wall.

In an example, the housing can electrode 508 can be electrically coupled to at least one other electrode (e.g., the first electrode 16A), or the housing can electrode 508 can be electrically isolated from other electrodes and capable of independent control. In an example, any of the first electrode 16A through the eighth electrode 66C can include at least one of a coil-type electrode, a ring-type electrode, or a tip electrode.

In an example, the right ventricular apex lead 15 can be configured to electrically couple the IMD 105 to at least one of the right ventricle 560, the right atrium 570, or the superior vena cava using at least one electrode (e.g., the first electrode 16A, the second electrode 16B, the third electrode 16C, or the fourth electrode 16D), the left ventricular lead 35 can be configured to electrically couple the IMD 105 to the left ventricle 565 using at least one electrode (e.g., the fifth electrode 36A or the sensor 36B), or the right ventricular septum lead 65 can be configured to electrically couple the IMD 105 to the interventricular septum using at least one electrode (e.g., the sixth electrode 66A, the seventh electrode 66B, or the eighth electrode 66C). In an example, at least one of the second electrode 16B, the third electrode 16C, or the fourth electrode 16D, can be configured to be located in, on, or near a right apical region of the heart 102. In an example, the fifth electrode 36A or the sensor 36B can be configured to be located in, on, or near a left apical region of the heart 102 or a left ventricular free lateral wall of the heart 102. In an example, a cardiac rhythm management device capable of delivering defibrillation energy can include a shocking electrode, such as the first electrode 16A, such as electrically tied or coupled to the housing can electrode.

FIG. 5 illustrates generally an example of several natural conduction systems of the heart. For example, a His bundle 521 can be coupled to the left branch bundle 522 and the right branch bundle 523. Left and right branch bundles can lead to Purkinje fibers 524 near an apex of the heart 102, such as on the left and right sides of the heart 102. In an example, the eighth electrode 66C can be located at or near the His bundle 521 or the AV node 520.

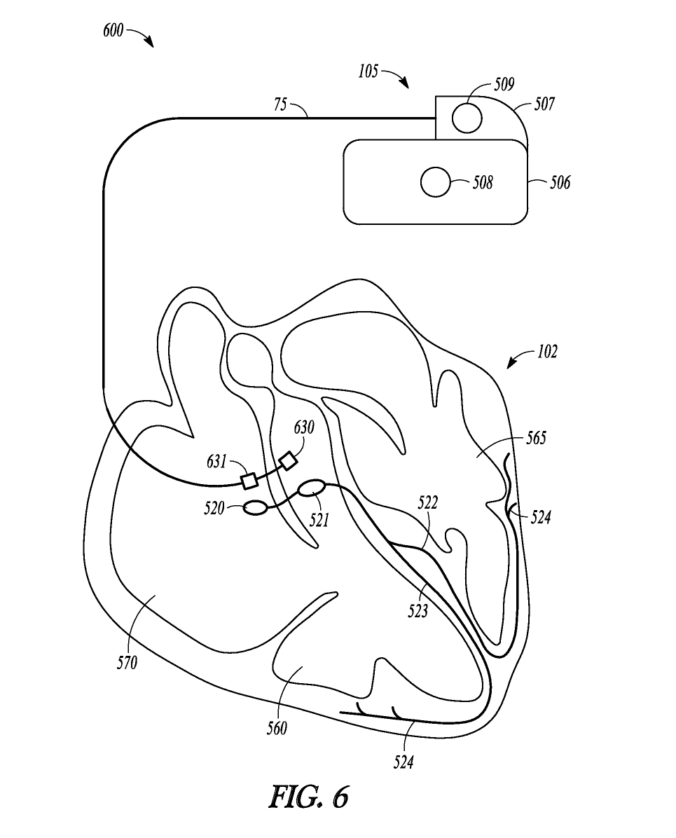

FIG. 6 illustrates generally an atrial lead 75, coupled to a tip electrode 630 and a ring electrode 631. The tip electrode 630 and ring electrode 631 can be disposed in the right atrium, such as at or near the His bundle 521, and can be configured to deliver electrostimulation energy to the His bundle 521. In an example, the atrial lead 75 can be used as a sensor, such as to provide information about a physical displacement of at least a portion of the atrial lead 75 in the subject 101. For example, the atrial lead 75 can be electrically coupled to the electrical energy delivery circuit 110 and the detector circuit 111, such as can be included in the IMD 105. The electrical energy delivery circuit 110 can be configured to provide a first signal to the atrial lead 75. The detector circuit 111 can be configured to receive and interpret a second signal in response to the first signal, and the second signal can be indicative of a physical displacement of the atrial lead 75, such as described by Ingle, in U.S. Patent Application No. 61/359,430 entitled LEAD MOTION SENSING VIA CABLE MICROPHONICS, which is hereby incorporated by reference in its entirety. A system can thus be deployed to provide electrostimulation to the heart 102, such as at or near a para-Hisian region, and to provide a cardiac diagnostic indication, such as an indication of cardiac capture, such as using a single lead.

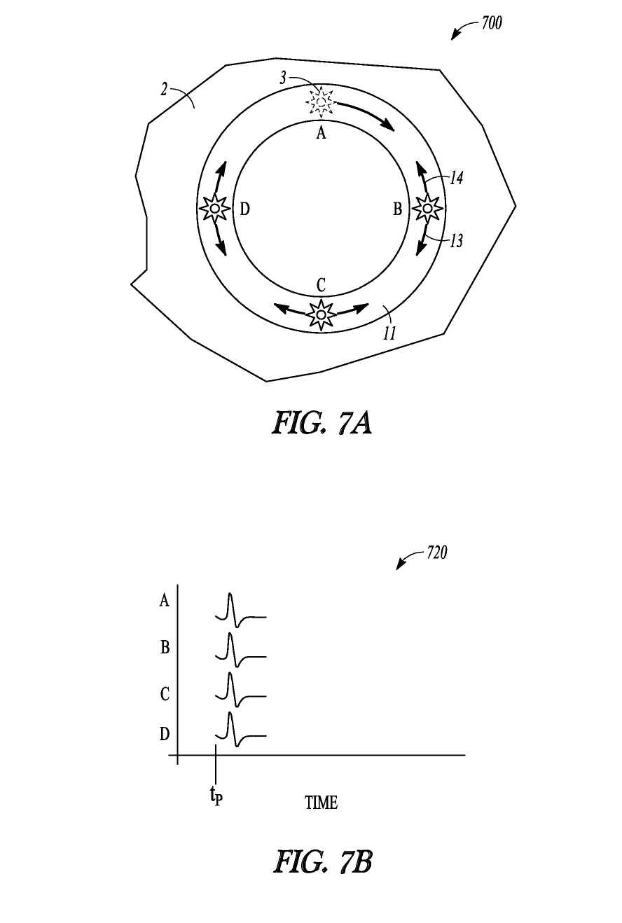

In an example, the systems described in FIGS. 2 through 6, among other systems that can be configured to provide or deliver electrostimulation to a patient body, can be used to identify or provide therapy for re-entrant cardiac activity, such as described in FIGS. 1A-1C. For example, FIG. 7A illustrates generally an example that can include disrupting a re-entry loop in cardiac tissue. In an example, the impulse 3 can be initiated in the myocardial tissue 2 of the heart tissue. The impulse 3 can follow a conduction pathway, such as a slowed pathway, and can cause an ectopic event, such as a PVC (see, for example, the discussion of FIG. 1A, above). In an example, an electrostimulation can be delivered in response to the detected impulse 3, such as using the electrical energy delivery circuit 110. In an example, the delivered electrostimulation can activate, or cause to be activated, other impulses (e.g., at locations B, C, or D, among other locations), such as to disrupt transmission or propagation of the impulse 3 along the re-entrant loop 11.

In an example, in response to an electrostimulation (e.g., an electrostimulation delivered at one or more of the locations B, C, or D, among other locations), one or more portions of the myocardial tissue 2 can be activated. For example, an electrostimulation can activate an impulse along the myocardial tissue 2 in multiple directions, such as along a second direction 13, or a third direction 14, such as to prevent re-entrant loop formation, or to disrupt a re-entrant loop. In an example, an electrostimulation can be delivered to the para-Hisian region (e.g., at or near the His bundle 521 or the AV node 520), such as to stimulate a His bundle or to activate natural conduction mechanisms of the heart, such as the Purkinje system, such as to disrupt a re-entrant pathway (e.g., a re-entrant pathway distal to the His bundle). In an example, an electrostimulation delivered to the para-Hisian region can cause some or all of the myocytes around a re-entrant loop to be stimulated, such as substantially concurrently or after a delay commensurate with a natural conduction impulse.

FIG. 7B illustrates generally an example of a chart 720 that can illustrate a coordinated response of portions of the myocardial tissue 2. For example, at time t_{P}, an electrostimulation can be provided to the myocardium, such as can be provided to tissue at any one or more of the locations A, B, C, or D, among others. In an example, at t_{P}, an electrostimulation can be provided to the para-Hisian region. In the example of the chart 720, an impulse can be delivered to, or propagate from, any one or more of the locations A, B, C, or D, among others, such as substantially concurrently.

In an example, upon detection of an ectopic event (e.g., using the detector circuit 111), an electrostimulation can be initiated at time t_{P}. In response, an electrostimulation can be provided to the myocardial tissue 2, such as at one or more of the locations B, C, or D, such as to disrupt re-entrant activity or an ectopic event. In an example, an electrostimulation can be provided to the para-Hisian region, and, in response, the myocardial tissue 2, such as at or near one or more of the locations A, B, C, or D, among other locations, can be activated using the heart's natural conduction pathways to disrupt or prevent re-entrant activity. In an example, an electrostimulation (e.g., an electrostimulation delivered to the para-Hisian region or to the myocardium, such as at the location B) can be provided using the IMD 105.

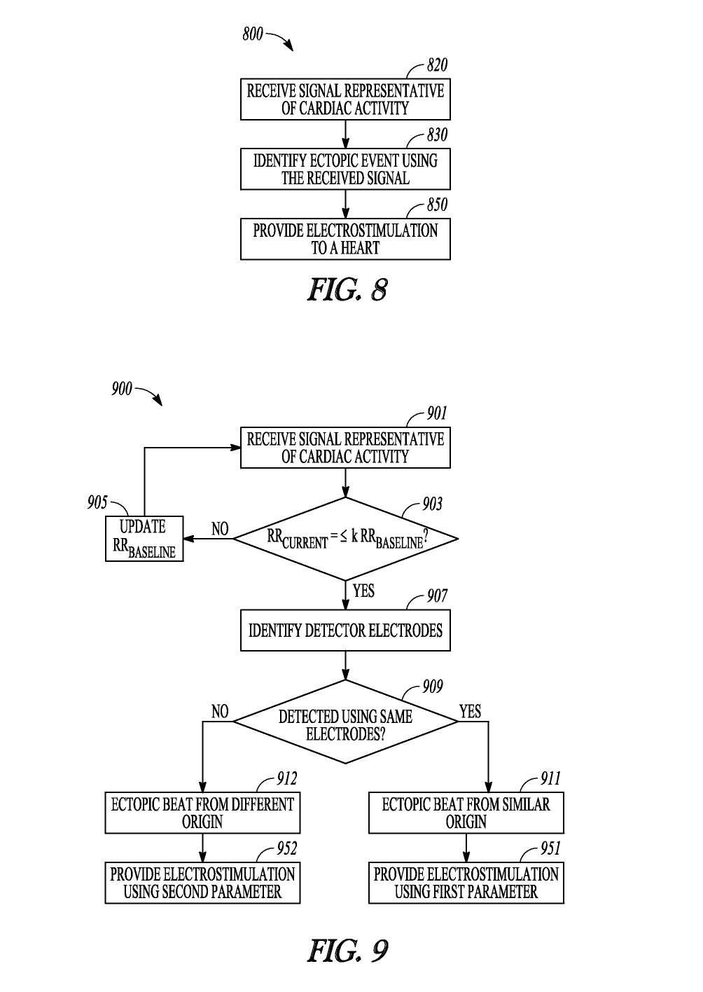

FIG. 8 illustrates generally an example that can include providing electrostimulation to a heart in response to an ectopic event. At 820, a signal representative of cardiac activity can be received, such as using the detector circuit 111. In an example, the signal representative of cardiac activity can be an electrical signal, such as can be obtained using at least one of an electrode (e.g., an implantable, surface, or external electrode), accelerometer, microphone, or other physiological sensor, such as can be used to receive cardiac activity information. In an example, the cardiac activity information can be received using, among other systems or devices, the system 500.

In an example, a signal representative of cardiac activity (e.g., the signal representative of cardiac activity received at 820) can include electrogram information, EKG information, or echocardiogram information, among other information. The information can be used to identify, among other things, normal sinus cardiac activity or irregular, dysfunctional cardiac activity. In an example, the signal representative of cardiac activity can be received and monitored over time, such as to monitor a subject's sinus rhythm. For example, the signal representative of cardiac activity can be received and monitored over time to identify a cardiac arrhythmia, such as a tachycardia episode (e.g., a ventricular tachycardia episode). In an example, a tachycardia episode can include an elevated heart rate that can sustain for an extended period, such as several seconds, minutes, or longer periods.

In an example, the signal representative of cardiac activity can be received and monitored to identify abnormalities in a sinus rhythm. For example, one or more ectopic events (e.g., an ectopic beat comprising a premature contraction in a ventricle or atrium) can be identified. In an example, an ectopic event can occur in the course of an otherwise normal sinus rhythm. For example, an ectopic event can occur adjacently sequentially to normal sinus heart beats, such as in the absence of a diagnosed tachyarrhythmia episode. In an example, a series of ectopic events can occur in the absence of a diagnosed tachyarrhythmia episode. In an example, a series of ectopic events can occur substantially sequentially, or one or more intervening sinus beats can occur among a series of ectopic events, such as in the absence of a diagnosed tachyarrhythmia episode.

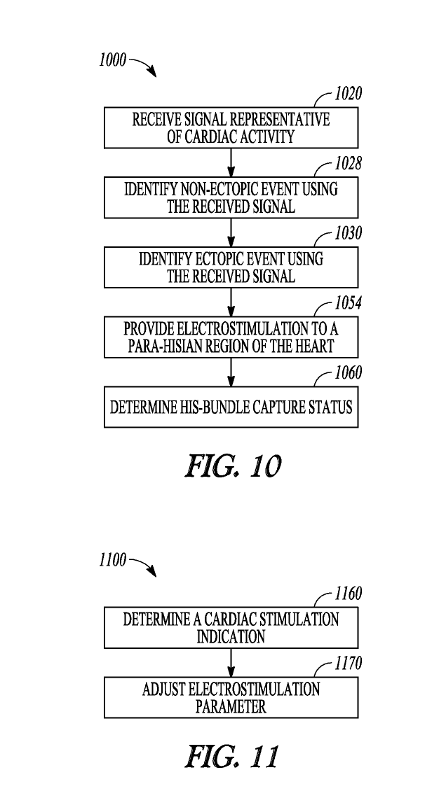

In an example, at 830, an ectopic event can be identified, such as using the cardiac activity information received at 820. In an example, an ectopic event can include a premature ventricular contraction or a premature atrial contraction, such as can occur sequentially and adjacently to an otherwise normal sinus beat. For example, the ectopic event identified at 830 can include a premature contraction event that can be sequentially adjacently preceded by a non-premature contraction event, such as illustrated in the example of FIG. 1C at 142. In an example, an ectopic event can be identified using the technique described below in the example of FIG. 9, among other techniques.

In an example, at 850, an electrostimulation can be provided to a heart, such as in response to identifying an ectopic event at 830. For example, the electrostimulation can be provided using the electrical energy delivery circuit 110 and delivered using the electrode 460. In an example, the electrical energy delivery circuit 110 can be configured to provide an electrostimulation to a para-Hisian region of the heart 102, such as using one or more of the tip electrode 630 or the ring electrode 631. In an example, the electrostimulation provided at 850 can comprise an electroshock or other pacing electrostimulation, such as in addition to a His bundle electrostimulation (e.g., substantially concurrently or sequentially). For example, the electrostimulation provided at 850 can include multiple electrostimulations comprising one or more of a His bundle electrostimulation, a right ventricular electrostimulation, a left ventricular electrostimulation, or a biventricular electrostimulation, among others.

FIG. 9 illustrates generally an example that can include identifying an ectopic event or providing electrostimulation using an origin-dependent parameter. In an example, an origin of one or more of a PVC or an ectopic event can be determined, such as using cardiac activity information.

In the example of FIG. 9, at 901, a signal representative of cardiac activity can be received, such as according to the discussion of FIG. 8 at 820. For example, the signal representative of cardiac activity can include cardiac activity information, such as can be received using an accelerometer or one or more pairs of implantable or external electrodes. In an example, the cardiac activity information can be received using, among other things, the detector circuit 111 or the system 500. The received signal representative of cardiac activity can be analyzed to identify an ectopic event. In an example, the signal can be received and analyzed in real-time such that an indication of an ectopic event can be provided as the ectopic event occurs. In an example, the processor circuit 112 can be used to receive and analyze the signal in real-time.

In an example, at 903, an ectopic event can be identified, such as by comparing a current R-R interval to a baseline R-R interval. In an example, an R-R interval can include an interval between adjacent, or temporally nearby, R wave peaks, such as can be determined using the signal representative of cardiac activity received at 901. For example, the magnitude of a received EKG or electrogram signal can be used to indicate an R-R interval, such as by identifying an interval between a first threshold magnitude crossing and a subsequent second threshold magnitude crossing of the received signal. In an example, a current or recent R-R interval (RR_{CURRENT}) can be compared to a baseline R-R interval (RR_{BASELINE}). In an example, if RR_{CURRENT} is less than or about equal to RR_{BASELINE}, then an ectopic event can be identified.

In an example, if RR_{CURRENT} is less than or about equal to a predetermined portion of RR_{BASELINE}, then an ectopic event can be identified. For example, if RR_{CURRENT} is less than or about equal to a value k*RR_{BASELINE}, and k=0.8, then an ectopic event can be identified when a current R-R interval duration differs from a baseline R-R interval by more than about 20%. The variable k can be specified to provide an appropriate threshold for determining the timing of an ectopic event, and can be patient- or application-specific. In an example, at 905, RR_{BASELINE} can be optionally updated, such as manually or automatically, if RR_{CURRENT} is not less than or equal to RR_{BASELINE}.

In an example, one or more electrode pairs can be used to detect an ectopic event. At 907, an electrode pair used to receive a signal representative of cardiac activity can be identified. For example, a first electrode pair (e.g., the unipolar electrode pair comprising the fourth electrode 16D and the can electrode 508) can receive a first signal representative of cardiac activity, and a second electrode pair (e.g., the bipolar electrode pair comprising the sixth electrode 66A and the seventh electrode 66B), can receive a second signal representative of cardiac activity. In an example, the first and second signals can be analyzed (e.g., at 903) to identify an ectopic event. The first and second signals can be compared, for example, to determine which signal indicates an earliest detection of the ectopic event. In an example, the first cardiac activity information can indicate the ectopic event occurred at a first time, and the second cardiac activity information can indicate that the same ectopic event occurred at a later second time. In an example, the first time can precede the second time, and the ectopic event can be localized to a region of the heart 102 nearer the first electrode pair than the second electrode pair. In an example, timing information received from three or more electrode pairs can be used to precisely identify a location of the ectopic event, such as using triangulation.

In an example, a first ectopic event can be detected, and a subsequent second ectopic event can be detected, such as according to the example of FIG. 9. In an example, the subsequent second ectopic event can occur within a first interval of the first ectopic event such that the first and second ectopic events can be considered to be the same ectopic event or at least two ectopic events in a single ectopic episode. In an example, the subsequent second ectopic event can occur after the first interval such that the first and second ectopic events are not considered to be in the same ectopic episode.

In the example of FIG. 9, at 909, information about a first ectopic event and information about a second ectopic event can be compared, such as to determine whether the first and second ectopic events were detected using the same electrode configuration (e.g., the same electrode pair). In an example, at 911, the first and second ectopic events can be first detected using the same electrode configuration. For example, although multiple electrode pairs can be used to detect the same ectopic events, when a particular electrode configuration can be used to first detect a first ectopic event and a subsequent second ectopic event, a common or similar origin of the first and second ectopic events can be indicated. At 912, a different or dissimilar origin can be indicated, such as when the first and second ectopic events are first detected using different electrode configurations (e.g., the first ectopic event can be first detected using a first electrode pair, and the second ectopic event can be first detected using a different second electrode pair).

In an example, at 951, an electrostimulation can be provided, such as using the electrical energy delivery circuit 110 to provide an electrostimulation to a para-Hisian region of the heart 102. In an example, the electrostimulation can be provided using a first electrostimulation parameter. For example, the first electrostimulation parameter can be used when a first and a different second ectopic event have a similar origin (e.g., as can be indicated at 911). The electrostimulation parameter can be used to determine at least one electrostimulation signal feature, such as an electrostimulation waveform shape, magnitude, location, or timing, among other features. In an example, an electrostimulation signal feature can be adjusted using the electrostimulation parameter. For example, a first electrostimulation parameter can be used to determine one or more electrostimulation signal features, such as can describe an electrostimulation configured to be provided in response to a first, origin-specific ectopic event. A second electrostimulation parameter can be used to determine one or more different electrostimulation signal features, such as can describe an electrostimulation configured to be provided in response to a second ectopic event having an origin that is not the same as the first ectopic event.

In an example, at 952, an electrostimulation can be provided to the heart 102, such as using the electrical energy delivery circuit 110. In an example, the electrostimulation can be provided using a different second electrostimulation parameter, such as can be indicated when the first and different second ectopic events do not have a similar origin (e.g., as can be indicated at 912). In an example, the second electrostimulation parameter can be used to determine an electrostimulation signal feature of a second, origin-specific electrostimulation signal.

In an example, some ectopic activity can have more than one foci, or one or more ectopic foci can travel along the myocardium. In an example, more than one electrode pair can be used to receive signals representative of cardiac activity, and the information received from the more than one electrode pair can be analyzed to determine one or more foci of ectopic events, such as using triangulation. In an example, an electrostimulation parameter can be adjusted, such as according to the foci of the ectopic events. For example, a first electrostimulation (e.g., comprising a first electrostimulation waveform or amplitude) can be provided in response to an indication of multiple ectopic foci in the left ventricle. In an example, a different second electrostimulation (e.g., comprising a different second electrostimulation waveform or amplitude) can be provided in response to an indication of a single foci in the right ventricle.

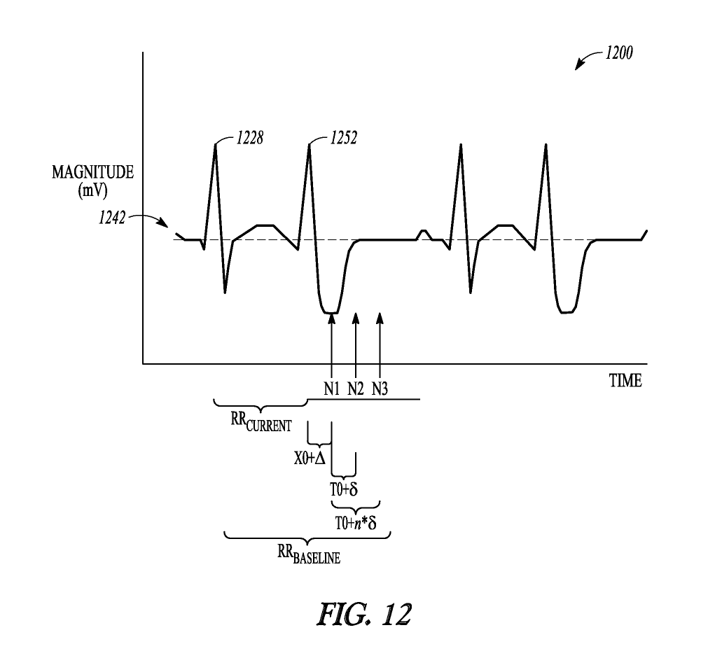

FIG. 10 illustrates generally an example that can include determining a His bundle capture status. At 1020, a signal representative of cardiac activity can be received, such as according to the discussion of FIG. 8 at 820. In an example, at 1028, a non-ectopic event can be identified, such as using the signal received at 1020. For example, a non-ectopic event can include a normal sinus event. In an example, a non-ectopic event can include a contraction event that follows a sinus event by an R-R interval of about RR_{BASELINE}. Features or characteristics of the signal representative of cardiac activity other than R-R interval, such as QRS width or phase, among others, can be used to identify a non-ectopic event at 1028.

In an example, at 1030, an ectopic event can be identified, such as according to the discussion of FIG. 9, among other techniques. In an example, the ectopic event can be identified at 1030 using the detector circuit 111 or the processor circuit 112, such as using information about cardiac activity. In an example, information about cardiac activity can be analyzed substantially in real-time by the processor circuit 112, such as to identify an onset of an ectopic event soon after the event occurs. In an example, a single ectopic event can be identified at 1030, such as subsequent to a non-ectopic event. For example, a non-ectopic event (e.g., a non-premature ventricular contraction) can adjacently sequentially precede the ectopic event (e.g., a PVC event).

In an example, at 1054, the processor circuit 112 can be configured to provide electrostimulation to a para-Hisian region of the heart 102. In an example, the electrostimulation can be provided to the para-Hisian region using at least partially overlapping first and second electrostimulation signal components, such as in opposite polarity from each other with respect to a reference component. Other electrostimulation signals can be used as well, such as comprising non-overlapping biphasic or monophasic waveforms.

In an example, at 1060, a His bundle capture status can be determined. For example, information received using His bundle sense electrode (e.g., one or more of the sixth electrode 66A, seventh electrode 66B, or eighth electrode 66C) or an RV apical sense electrode can be used to receive information about His bundle activity. In an example, other systems or methods can be used to determine the His bundle capture status. For example, Maskara et al., U.S. Provisional Patent Application No. 61/452,412, entitled HIS CAPTURE VERIFICATION USING ELECTRO-MECHANICAL DELAY, which is hereby incorporated herein by reference in its entirety, refers to using timing information about cardiac activity to discriminate between Purkinje fiber cardiac capture, cell-to-cell conduction cardiac capture, and intrinsic conduction cardiac contractions. In an example, Dong et al., U.S. patent application Ser. No. 13/094,416, entitled HIS BUNDLE CAPTURE VERIFICATION AND MONITORING, which is hereby incorporated herein by reference in its entirety, refers to using information about a detected QRS complex to identify His bundle capture. These systems or methods, among others, can be used to identify the His bundle capture status at 1060.

In an example, at 1060, a cardiac function status can be determined, such as before or after determining the His bundle capture status. In an example, the signal representative of cardiac activity can be continuously monitored, such as after providing the electrostimulation to the para-Hisian region (e.g., the electrostimulation provided at 1054). The signal representative of cardiac activity can be used to identify a heart rate, or a cardiac arrhythmia, such as a tachycardia episode, among other things. In an example, the signal representative of cardiac activity can be used to identify one or more subsequent ectopic events, such as having different origins. In an example, a pre-electrostimulation cardiac rhythm can be compared to a post-electrostimulation cardiac rhythm, such as using the processor circuit 112 to identify a cardiac rhythm change or to provide the cardiac function status. In an example, one or more electrostimulations can be indicated, such as according to the cardiac function status.

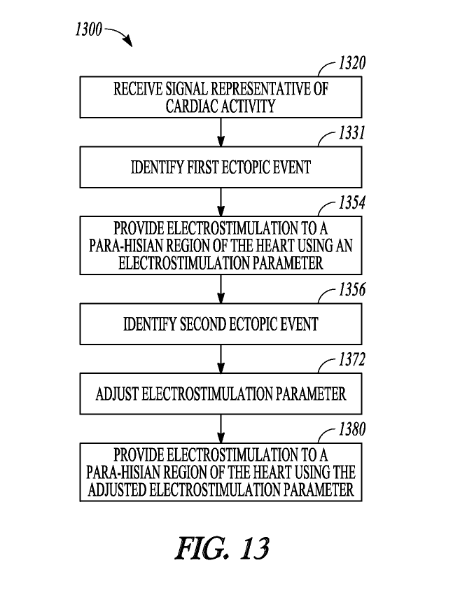

FIG. 11 illustrates generally an example that can include adjusting an electrostimulation parameter. For example, at 1160, a cardiac stimulation indication can be determined. In an example, the cardiac stimulation indication can be determined subsequent to, or in response to, providing electrostimulation to the heart 102, such as an electrostimulation provided to a para-Hisian region of the heart 102 (see, e.g., FIG. 10 at 1054). In an example, the cardiac stimulation indication can include at least one of a His bundle capture indication, or other cardiac capture indication. For example, the cardiac stimulation indication can include an indication of ventricular or atrial activity, such as in response to a His bundle electrostimulation or other electrostimulation (e.g., a ventricular pacing electrostimulation). In an example, the cardiac stimulation indication can include an indication of Purkinje fiber conduction, such as in response to a His bundle electrostimulation.

In an example, the cardiac stimulation indication determined at 1160 can indicate at least one of cardiac tissue capture, His bundle capture, or other cardiac tissue activity information. In an example, when the cardiac stimulation indication does not indicate at least one of cardiac tissue capture, His bundle capture, or other cardiac tissue activity, such as in response to an electrostimulation, an electrostimulation parameter change can be indicated.

In an example, at 1170, an electrostimulation parameter can be adjusted. In an example, an electrostimulation parameter can be used to determine at least one feature of an electrostimulation. For example, an electrostimulation parameter can indicate an electrostimulation signal that includes a particular electrostimulation waveform shape, magnitude, location, delay, or phase, among other features. In an example, an electrostimulation parameter can indicate how many electrostimulations to deliver, or an interval of electrostimulation delivery.

At 1170, at least one electrostimulation parameter can be adjusted, such as corresponding to at least one waveform feature. For example, an electrostimulation waveform shape can be adjusted (e.g., from a square wave pulse to a triangle wave pulse), or an electrostimulation magnitude can be adjusted (e.g., increased or decreased), such as in response to a determined cardiac stimulation indication that indicates non-capture of cardiac tissue. In an example, a number of electrostimulation deliveries can be adjusted, or an interval between electrostimulation deliveries can be adjusted. In an example, at 1170, adjusting an electrostimulation parameter can include adjusting multiple electrostimulation parameters, such as before a subsequent electrostimulation.

In an example, such as including two or more detected ectopic events, adjusting an electrostimulation parameter at 1170 can be conditioned on at least one of His capture or other cardiac response to a previous electrostimulation. For example, when a cardiac response indicates that a previous electrostimulation was ineffective to prevent a subsequent ectopic event, an electrostimulation parameter can be adjusted even though some cardiac tissue may have been captured by the electrostimulation. In an example, an electrostimulation parameter can be adjusted at 1170 when a most-recent ectopic event indicates a different characteristic than the first ectopic event. For example, an electrostimulation parameter can be adjusted when a first ectopic event indicates a different morphology, such as a different coupling interval to the preceding sinus beat, or a different origin, than a subsequent second ectopic event.

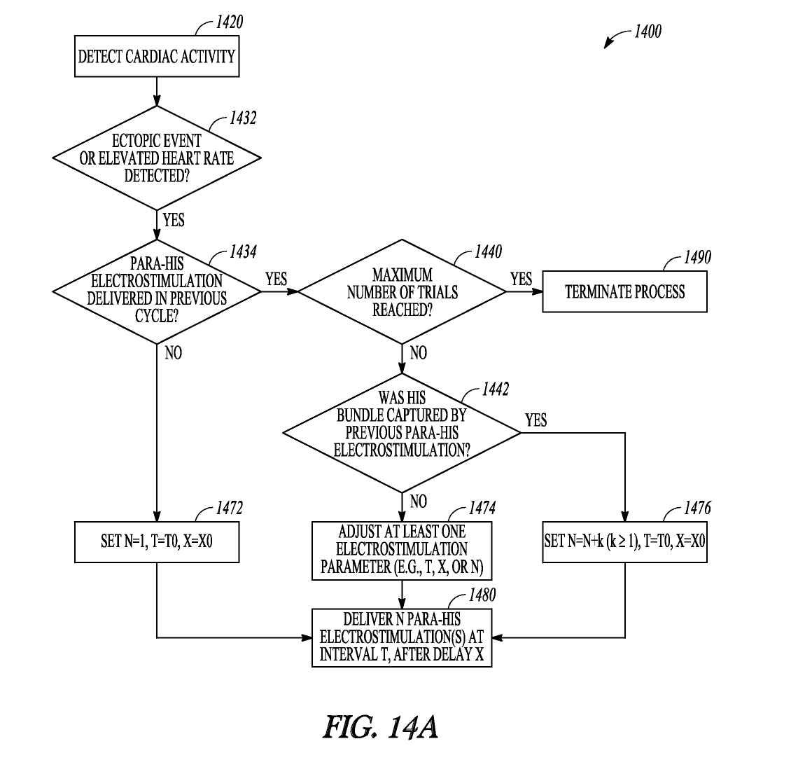

FIG. 12 illustrates generally an example of a chart 1200 that can include a waveform 1242 (e.g., an electrogram waveform) indicative of cardiac electrical activity of the heart 102. In an example, the waveform 1242 can include an indication of a first cardiac event, such as a sinus event 1228. The first sinus event 1228 can be identified, such as according to the discussion of FIG. 10 at 1028. A first ectopic event 1252 can occur, such as subsequent to the sinus event 1228, such as after an interval of about RR_{CURRENT}. In an example, RR_{CURRENT} can be less than a baseline interval of about RR_{BASELINE}, or an expected interval for normal sinus events. In an example, the sinus event 1228 can adjacently sequentially precede the first ectopic event 1252. In an example, the first ectopic event 1252 can be identified, such as according to the discussion of FIG. 10 at 1030.

In an example, a first electrostimulation N1 can be provided to the His bundle 521 (e.g., according to the discussion of FIG. 10 at 1054, such as using the electrical energy delivery circuit 110). In an example, the first electrostimulation

N1 can be provided after a delay X0+Δ from an onset of the first ectopic event 1252. In an example, X0 can indicate a specified or measured baseline delay, and Δ can indicate a specified change to the baseline delay, either positive or negative. In an example, the processor circuit 112 can receive information about cardiac activity, identify an ectopic event using the received information, and initiate delivery of an electrostimulation in response to the identified ectopic event, such as before the ectopic event terminates.

In the example of FIG. 12, a second electrostimulation N2 can be optionally provided, such as following the first electrostimulation N1. For example, if a cardiac stimulation indication does not indicate His bundle capture (see, e.g., FIG. 11 at 1160), such as in response to a first electrostimulation of a His bundle, the second electrostimulation N2 can be provided. For example, some cardiac cells can be in a refractory period (e.g., the cells can be unreceptive to electrostimulation) at or near the time a first electrostimulation can be delivered. In an example, a second subsequent electrostimulation, such as can be delivered or provided after a delay, can be used to capture the cells. In an example, a number of electrostimulations (e.g., N1, N2, etc.) can be increased, such as until cardiac capture can be verified or until a specified number of electrostimulations is reached.

In an example, the second electrostimulation N2 can be provided after an interval T0+δ, such as measured from delivery of the first electrostimulation N1. In an example, T0 can indicate a specified or measured baseline interval, and δ can indicate a specified change to the baseline interval, either positive or negative. In an example, a third electrostimulation N3 can be provided. The third electrostimulation N3 can be provided after an interval T0+n*δ, where n can be specified. In an example, δ, Δ, or n can be specified, such as according to patient-specific or other experimental data. In the example of FIG. 12, any one or more of the parameters X0, T0, Δ, δ, n, or the number of electrostimulations N1, N2, N3, etc., can be adjusted (e.g., at 1170) such as automatically using the processor circuit 112 or manually using the external module 115.

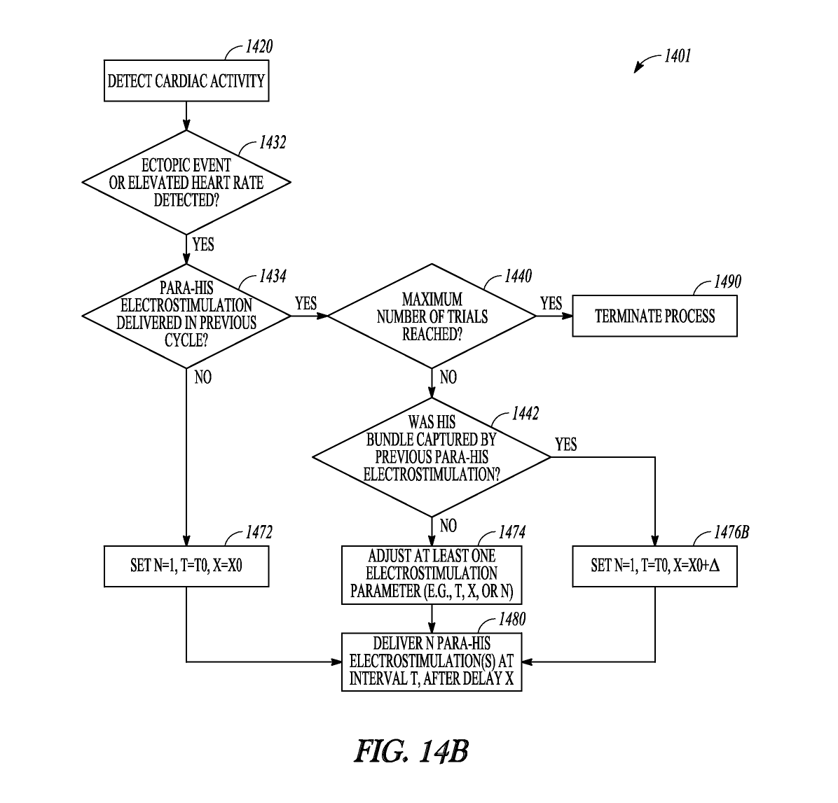

FIG. 13 illustrates generally an example 1300 that can include providing a first electrostimulation to a para-Hisian region, adjusting an electrostimulation parameter, and providing a second electrostimulation to the para-Hisian region. At 1320, a signal representative of cardiac activity can be received, such as according to the discussion of FIG. 10 at 1020. At 1331, a first ectopic event can be indentified, such as according to the discussion of FIG. 10 at 1030. At 1354, an electrostimulation can be provided to a para-Hisian region of the heart 102, such as using an electrostimulation parameter. In an example, the electrostimulation parameter can identify how many electrostimulations to provide to the para-Hisian region, or one or more features of the electrostimulation signal. In an example, the electrostimulation can be provided according to the discussion of FIG. 8 at 850.

In an example, at 1356, a second ectopic event can be identified. For example, the second ectopic event can be identified subsequent to the first ectopic event (e.g., the first ectopic event identified at 1331), such as within a specified number of intervening sinus events, or within a specified interval. In an example, the second ectopic event can be adjacent sequentially to a preceding non-ectopic event. In an example, the second ectopic event can be identified according to the discussion of FIG. 9.

In an example, at 1372, an electrostimulation parameter can be adjusted, such as in response to identifying the second ectopic event. The electrostimulation parameter can be adjusted according to the discussion of FIG. 11 at 1170. In an example, an electrostimulation parameter, such as the electrostimulation parameters of FIG. 12, can be adjusted at 1372.

In an example, at 1380, electrostimulation can be provided to the para-Hisian region of the heart 102, such as using the adjusted electrostimulation parameter. For example, the electrostimulation can be provided according to the discussion of FIG. 10 at 1054.

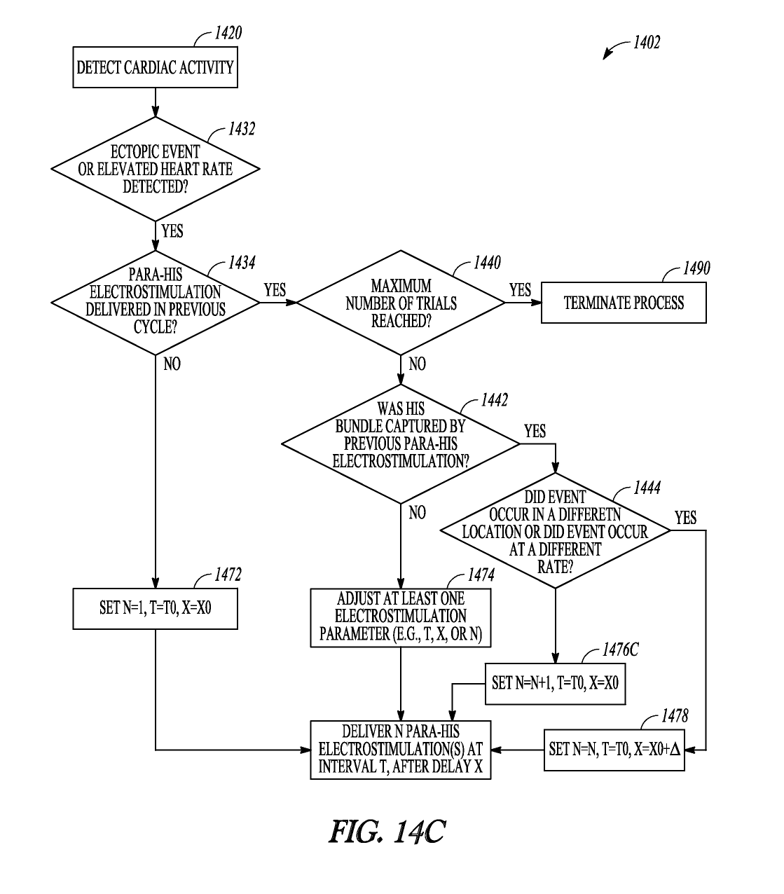

FIG. 14A illustrates generally an example 1400 that can include increasing a number of electrostimulations, such as in response to non-capture of the His bundle 521. In an example, at 1420, cardiac activity can be detected, such as using the detector circuit 111 or the processor circuit 112. In an example, electrodes or other physiological sensors, such as disposed on or in the subject 101, can be used to detect the cardiac activity. In an example, an electrode pair can be used to detect information about cardiac electrical activity, such as can be used to provide an indication of an ectopic event (e.g., according to the discussion of FIG. 9).

In an example, at 1432, an ectopic event or elevated heart rate can be detected, such as using the detector circuit 111 or the processor circuit 112. In an example, detecting the ectopic event or elevated heart rate at 1432 can include identifying ventricular bigeminy or trigeminy, such as to provide an indication of a pattern of ectopic or premature ventricular activity. For example, a single benign ectopic event can be untreated, but a series of two or more ectopic events can indicate a need for therapy. In an example, one or more detected ectopic events, or an elevated heart rate, can be in the absence of a diagnosed tachyarrhythmia episode.

At 1434, such as upon detection of at least one of an ectopic event or an elevated heart rate, a para-His electrostimulation status can be determined. For example, the para-His electrostimulation status can include information about whether a para-His electrostimulation was provided in response to a previously detected ectopic event or elevated heart rate, such as within a specified interval of a current ectopic event or elevated heart rate episode. In an example, if a para-His electrostimulation was not previously provided, a baseline electrostimulation parameter can be set. For example, at 1472, a number of electrostimulations N can be set to 1, an electrostimulation interval T can be set to T0 (e.g., 0 ms), and an electrostimulation delay X can be set to X0 (e.g., 1 ms). In an example, after establishing a baseline electrostimulation parameter, an electrostimulation can be provided to the para-Hisian region at 1480, such as according to the parameters N, T, or X, among other parameters. For example, N para-Hisian electrostimulations can be provided, such as after a delay X, and, for N>1, the electrostimulations can be temporally spaced at an interval T. In an example, one or more intervals between electrostimulations can be adjusted. For example, a first interval T1 between first and second electrostimulations can be a first duration (e.g., T1=T0+δ), and a second interval T2, such as between second and third electrostimulations, can be a different second duration (e.g., T2=T0+2δ).

In an example, at 1440, if a para-His electrostimulation was previously provided, such as determined at 1434, the processor circuit 112 can evaluate whether a maximum number of trials has been reached. For example, if a threshold number of trials has been exceeded, at 1490 the electrostimulation process can be terminated. In an example, if a threshold number of trials has been exceeded, a cardiac dysfunction other than an ectopic event or ectopic episode can be indicated. For example, the subject 101 can be experiencing a tachyarrhythmia episode, and other treatments or therapies can be pursued (e.g., using the IMD 105).

In an example, at 1440, if the maximum number of trials is not reached, further analyses can be performed. For example, at 1442, the processor circuit 112 can evaluate whether the His bundle was effectively captured by a previous para-His bundle electrostimulation. In an example, if the His bundle was effectively captured by a previous electrostimulation, the electrostimulation may not have been sufficient to disrupt re-entrant activity (e.g., the re-entrant loop 11). At 1476, the number of electrostimulations can be increased, such as to attempt to disrupt or eliminate the re-entrant activity. In an example, the number of electrostimulations N can be increased by k electrostimulations, such as by one or more electrostimulations. In an example, the number of electrostimulations can be increased from one to two or more, such as can be delivered at an interval T0 (e.g., for T0 greater than 0seconds). In an example, after adjusting one or more electrostimulation parameters at 1476, an electrostimulation can be provided to the para-Hisian region at 1480, such as according to the parameters N, T, or X, among other parameters.

In an example, an ineffective electrostimulation can be indicated when the His bundle was not captured by a previous electrostimulation. Accordingly, at 1474, one or more electrostimulation parameter can be adjusted. For example, T, X, or N can be adjusted, or a waveform shape, amplitude, polarity, or phase, among other features, can be adjusted. In an example, after adjusting the electrostimulation parameters at 1474, an electrostimulation can be provided to the para-Hisian region at 1480, such as according to the parameters N, T, or X, among other parameters.

FIG. 14B illustrates generally an example 1401 that can include adjusting a para-Hisian electrostimulation delay or latency. In an example, an electrostimulation parameter X can include a delay interval or latency, such as a delay between a detected ectopic event (e.g., an onset of the ectopic event, or a magnitude threshold crossing of a signal indicative of the ectopic event) and an initiation of a para-Hisian electrostimulation, such as in response to the detected ectopic event.

In an example, at 1442, the processor circuit 112 can evaluate whether the His bundle was effectively captured by a previous para-His electrostimulation. In an example, if the His bundle was effectively captured by the previous electrostimulation, the electrostimulation may not have been sufficiently effective to disrupt re-entrant activity (e.g., the re-entrant loop 11). At 1476B, the delay before the first electrostimulation can be increased or decreased, such as to attempt to disrupt or eliminate re-entrant activity. In an example, the delay can be increased by 1 ms (e.g., the initial delay X=X0=1 ms can be increased to X=X0+1 ms=2 ms). In an example, such as after adjusting the delay electrostimulation parameter at 1476B, an electrostimulation can be provided to the para-Hisian region at 1480, such as according to the parameters N, T, or X, among other parameters.

FIG. 14C illustrates generally an example 1402 that can include adjusting an electrostimulation parameter when ectopic events are detected at different origins or different rates. In an example, a first ectopic event can occur at a first location in the myocardium, and a second ectopic event can occur at a different second location in the myocardium. In an example, an ectopic event location can be determined or differentiated, such as according to the discussion of FIG. 9. In an example, a series of ectopic events can be detected, and an electrostimulation can be provided in response to one or more of the ectopic events. In an example, a first ectopic event can be detected at a first location, and in response, a first electrostimulation can be provided, such as using a first electrostimulation parameter. In an example, a second ectopic event, such as subsequent to the first ectopic event, can be detected at a different second location, and in response, a second electrostimulation can be provided, such as using a second electrostimulation parameter. In an example, a third ectopic event, such as subsequent to the first and second ectopic events, can be detected, such as at the first location. In response to the third ectopic event, a third electrostimulation can be provided, such as using a third electrostimulation parameter.

In an example, at 1442, the processor circuit 112 can evaluate whether the His bundle was effectively captured by a previous para-His bundle electrostimulation. In an example, if the His bundle was effectively captured by the previous electrostimulation, the electrostimulation may not have been sufficiently effective to disrupt re-entrant activity (e.g., the re-entrant loop 11). At 1444, the processor circuit 112 can evaluate location information, such as for a current ectopic event and a preceding ectopic event. Alternatively, or in addition, at 1444, the processor circuit 112 can evaluate an ectopic event rate (e.g., the processor circuit 112 can determine an interval between a current ectopic event and a preceding ectopic event).

In an example, if the current ectopic event occurs substantially proximally to the preceding ectopic event, or if the current ectopic event rate is greater than the preceding ectopic event rate by at least a threshold amount, then a first electrostimulation parameter can be adjusted. For example, at 1478, a delay interval can be increased from X=X0 to X=X0 +Δ. In an example, if the current ectopic event does not physically occur sufficiently proximally to the preceding ectopic event (e.g., determined using triangulation of signals received using the detector circuit 111), or if a current ectopic event rate is less than a preceding ectopic event rate, such as by at least a threshold amount, then a second electrostimulation parameter can be adjusted. For example, at 1476C, a number of electrostimulations can be increased, such as from N=1 to N=2. In an example, such as after adjusting an electrostimulation parameter at 1476C or 1478, an electrostimulation can be provided to the para-Hisian region at 1480, such as according to the parameters N, T, or X, among other parameters.

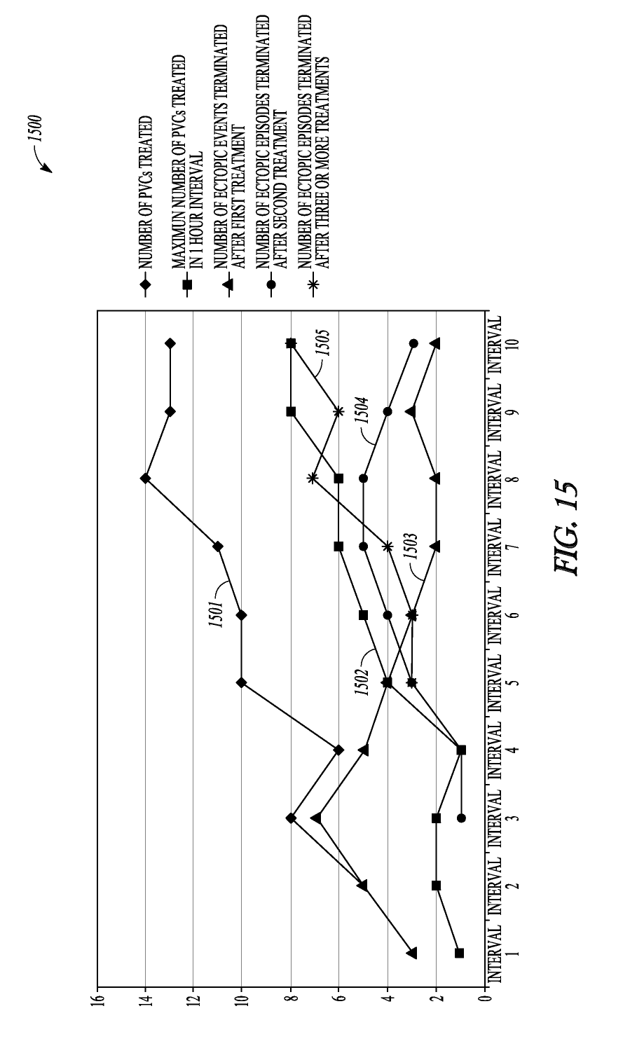

FIG. 15 illustrates generally a chart 1500 that can include information about an electrostimulation trend or a cardiac response trend. For example, the chart 1500 can include, such as along an x axis, intervals indicative of data collection intervals. For example, a first interval (e.g., INTERVAL 1) can include information collected during a first 24 hour period, or during some other interval (e.g., an hour, a week, etc.).

In an example, the chart 1500 can include a treated PVC trendline 1501. For example, the treated PVC trendline 1501 can illustrate graphically, for a particular patient or set of patients, a number of attempted PVC treatments (e.g., successful or unsuccessful treatments), such as for a given period. For example, the chart 1500 can indicate that about 10 PVCs were treated (e.g., with or without success) during each of the fifth and sixth intervals (e.g., during fifth and sixth days of data collection). The chart 1500 can indicate a trend, such as illustrating that a patient was treated for relatively few PVCs during a first number of intervals (e.g., less than 10 PVCs during intervals 1 through 4), but the patient was treated for more PVCs during later intervals (e.g., greater than 10 PVCs during intervals 7 through 10). In the example of FIG. 15, a clinician can use the treated PVC trendline 1501 to determine a progression of premature ventricular behavior, such as can be indicative of an underlying cardiac dysfunction.

In an example, information about a time of occurrence of treated and/or non-treated ectopic activity can be monitored or trended, such as using the chart 1500. In an example, a distribution of a time of occurrence of treated and/or non-treated ectopic activity can be monitored or trended, such as over the course of a pre-determined interval, such as an hour, a day, or a month, etc. For example, a number of treated ectopic events can be monitored or trended, or a number of non-treated ectopic events can be monitored or trended. In an example, time information about an interval between ectopic events, such as between treated events, non-treated events, or both, can be monitored or trended.

Other information about PVCs or ectopic activity can be trended. For example, the chart 1500 can include a trendline 1502, such as can indicate a maximum number of PVCs treated in a specified interval (e.g., a 1 hour interval). The trendline 1502 can be used to provide an indication of the frequency of PVC activity or how closely clustered PVC events can be. In an example, an increasing number of PVCs treated within a specified interval can indicate an extended ectopic episode, such as can be due to worsening heart failure or other conditions.

In an example, the chart 1500 can include a trendline 1503, such as can indicate a number of ectopic events terminated after a first treatment (e.g., after delivery of a first electrostimulation, such as can be provided to a para-Hisian region). For example, the trendline 1503 can provide an indication of a success rate for using electrostimulation to disrupt re-entrant activity. In the example of FIG. 15, such as during INTERVAL 9, for example, a number of PVCs treated within a particular interval can be relatively high (e.g., about 8 PVCs; see trendline 1502). The trendline 1503 can indicate that a relatively small number of these PVCs were successfully addressed or treated using the first electrostimulation. In the example of FIG. 15, less than half of the ectopic events that occurred during INTERVAL9were terminated after the first electrostimulation.

In an example, the chart 1500 can include a trendline 1504, such as can indicate a number of ectopic events terminated after a second treatment (e.g., after delivery of a first electrostimulation and a subsequent second electrostimulation, such as provided to the para-Hisian region using the same or different electrostimulation parameters). In an example, the chart 1500 can include a trendline 1505 indicative of a number of ectopic events terminated after a third treatment (e.g., after delivery of a first, a second, and a third electrostimulation, such as can be provided to the para-Hisian region, such as using first, second, and third electrostimulation parameters, respectively). In an example, the trendlines 1503, 1504, or 1505, among other trends, can be used to determine a severity of an ectopic episode.

In an example, other information about cardiac ectopic activity can be monitored or trended. For example, location information about cardiac ectopic activity can be monitored or trended, such as to identify a progression or propagation of cardiac ectopic activity, or to identify how often ectopic activity occurs in a particular region of cardiac tissue. In an example, location information about a particular ectopic episode can be monitored or trended, or location information about cardiac ectopic activity over longer periods of time can be monitored or trended. In an example, an electrostimulation therapy can be adjusted in response to an identified trend in cardiac ectopic activity location (see, for example, the discussion of FIG. 9 regarding using one or more different electrostimulation parameters depending on an identified origin of an ectopic beat).

### VARIOUS NOTES & EXAMPLES

Example 1 can include subject matter (such as an apparatus, such as can be implantable) that can comprise a detector circuit, an electrical energy deliver circuit, or a processor circuit. In an example, the detector circuit can be configured to receive a signal representative of electrical activity of a heart, and the electrical energy delivery circuit can be configured to provide an electrostimulation to be delivered to a location at or near a His Bundle of the heart. In an example, the processor circuit can be coupled to the detector circuit and the electrical energy delivery circuit, and the processor circuit can be configured to identify, in the absence of a diagnosed tachyarrhythmia episode, a first premature ventricular contraction (PVC) of the heart using the received signal representative of electrical activity of the heart and, in response to the identified first PVC, initiate delivery of the electrostimulation to be delivered to the location at or near the His bundle using the electrical energy delivery circuit.

In Example 2, the subject matter of Example 1 can optionally include a processor circuit, such as can be configured to identify, in the absence of a diagnosed tachyarrhythmia episode, an onset of the first PVC, and in response to the identified onset of the first PVC, initiate delivery of the electrostimulation to be delivered to the location at or near the His bundle during the first PVC.

In Example 3, the subject matter of one or any combination of Examples 1-2 can optionally include a processor circuit, such as can be configured to identify, in the absence of a diagnosed tachyarrhythmia episode, the first PVC, identify a non-premature ventricular contraction of the heart, the non-premature ventricular contraction adjacently sequentially precedes the first PVC, and, in response to the identified first PVC, initiate delivery of the electrostimulation to be delivered to the location at or near the His bundle before initiation of a refractory period resulting from the identified non-premature ventricular contraction of the heart.

In Example 4, the subject matter of one or any combination of Examples 1-3 can optionally include a processor circuit, such as can optionally be configured to determine a His bundle capture status in response to an electrostimulation delivered to a location at or near the His bundle.

In Example 5, the subject matter of one or any combination of Examples 1-4 can optionally include a processor circuit, such as can be configured to adjust an electrostimulation parameter in response to the determined His bundle capture status.

In Example 6, the subject matter of Example 5 can optionally include a processor circuit, such as can be configured to adjust at least one of an electrostimulation delay interval or how many electrostimulations are delivered in response to the determined His bundle capture status.

In Example 7, the subject matter of one or any combination of Examples 1-6 can optionally include a processor circuit, such as can be configured to determine an indication of a first origin of the first PVC of the heart, and in response to the identified first PVC, initiate delivery of the electrostimulation using the electrical energy delivery circuit and an origin-dependent electrostimulation parameter.

In Example 8, the subject matter of one or any combination of Examples 1-7 can optionally include an electrical energy delivery circuit, such as can be configured to provide the electrostimulation to a location in a right ventricle of the heart and on a septum at or near the His bundle.

In Example 9, the subject matter of one or any combination of Examples 1-8 can optionally include a processor circuit, such as can be configured to determine a cardiac stimulation indication in response to a delivery of the electrostimulation to the location at or near the His bundle.

In Example 10, the subject matter of one or any combination of Examples 1-9 can optionally include a processor circuit, such as can be configured to identify, in the absence of a diagnosed tachyarrhythmia episode, the first PVC of the heart using the received signal, and, in response to the identified first PVC, initiate delivery of a first electrostimulation to be delivered to the location at or near the His bundle using the electrical energy delivery circuit and an electrostimulation parameter. In an example, the processor circuit of Example 10 can optionally be configured to identify, in the absence of a diagnosed tachyarrhythmia episode and within a specified first time interval of the first PVC, a subsequent second PVC of the heart using the received signal, adjust the electrostimulation parameter, and, in response to the identified second PVC, initiate delivery of a second electrostimulation to be delivered to the location at or near the His bundle using the electrical energy delivery circuit and the adjusted electrostimulation parameter.

In Example 11, the subject matter of Example 10 can optionally include a processor circuit, such as can be configured to trend information about the first and second electrostimulations.

In Example 12, the subject matter of one or any combination of Examples 10-11 can optionally include a processor circuit, such as can be configured to determine a His bundle capture status in response to an electrostimulation delivered to a location at or near the His bundle.

In Example 13, the subject matter of Example 12 can optionally include a processor circuit, such as can be configured to determine the His bundle capture status in response to the first electrostimulation, and, when the His bundle capture status indicates non-capture, initiate delivery of the second electrostimulation to the location at or near the His bundle.

In Example 14, the subject matter of one or any combination of Examples 10-13 can optionally include a processor circuit, such as can be configured to determine a cardiac stimulation indication in response to at least one of the first electrostimulation or the second electrostimulation.

In Example 15, the subject matter of one or any combination of Examples 10-14 can optionally be configured to initiate delivery of the first electrostimulation to be delivered to the location at or near the His bundle, such as in response to the identified first PVC, wherein the first electrostimulation includes a first waveform shape, a first waveform amplitude, and a first waveform polarity. In an example, the processor circuit of Example 15 can optionally be configured to initiate delivery of the second electrostimulation to be delivered to the location at or near the His bundle, such as in response to the identified second PVC, wherein the second electrostimulation includes at least one of a different second waveform shape, a different second waveform amplitude, or a different second waveform polarity than was used with the first electrostimulation.

In Example 16, the subject matter of one or any combination of Examples 10-15 can optionally be configured to trend information about the first electrostimulation, the second electrostimulation, and at least one of a cardiac capture status or a His bundle capture status.

Example 17 can include subject matter (such as a method) comprising detecting, using a medical device, a first premature ventricular contraction (PVC) of a heart in the absence of a diagnosed tachyarrhythmia episode, and, in response to the detecting the first PVC, using the medical device, automatically delivering a first electrostimulation to a location at or near a His bundle.

In Example 18, the subject matter of Example 17 can optionally include delivering the first electrostimulation to the location at or near the His bundle, such as can optionally include delivering an electrostimulation to a location at or near at least one of a right ventricle of the heart or a left ventricle of the heart.

In Example 19, the subject matter of one or any combination of Examples 17-18 can optionally include detecting, using the medical device, a subsequent second PVC of the heart in the absence of a diagnosed tachyarrhythmia episode within a specified first time interval of detecting the first PVC, and, in response to the detecting the second PVC, adjusting an electrostimulation parameter of the medical device. Example 19 can optionally include delivering, using the medical device, a second electrostimulation to the location at or near the His bundle using the adjusted electrostimulation parameter. In Example 19, the adjusting the electrostimulation parameter can optionally include adjusting at least one of an electrostimulation delay interval or how many electrostimulations are delivered.

Example 20 can include subject matter (such as a method) comprising detecting, using a medical device, a first premature ventricular contraction (PVC) of a heart in the absence of a diagnosed tachyarrhythmia episode, detecting, using the medical device, an indication of a first origin of the first PVC of the heart, and delivering, using the medical device, a first electrostimulation to a location at or near a His bundle and in response to the detecting the first PVC. In an example, Example 20 can optionally comprise detecting, using the medical device, a subsequent second PVC of the heart in the absence of a diagnosed tachyarrhythmia episode and within a specified first time interval of detecting the first PVC of the heart, and detecting, using the medical device, an indication of a second origin of the second PVC of the heart. In an example, Example 20 can optionally comprise comparing the indication of the detected first origin and the indication of the detected second origin, and, when the indication of the detected second origin is about the same as indication of the first detected origin, delivering, using the medical device, a second electrostimulation to the location at or near the His bundle using a first electrostimulation parameter of the medical device. In an example, when the indication of the detected second origin differs from the indication of the detected first origin by greater than a threshold amount, Example 20 can optionally comprise delivering, using the medical device, the second electrostimulation to the location at or near the His bundle using a second electrostimulation parameter of the medical device.

These non-limiting examples can be combined in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer- or processor-readable medium, or other machine-readable medium, such as can be encoded with instructions operable to configure an electronic device to perform methods as described in the above examples. An implementation of such methods can include code, such as microcode, assembly language code, a higher-level language code, or the like. Such code can include computer-readable (or processor-readable or machine-readable) instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
a detector circuit configured to receive a signal representative of electrical activity of a heart;
an electrical energy delivery circuit configured to provide an electrostimulation to be delivered to a location at or near a His Bundle of the heart; and
a processor circuit, coupled to the detector circuit and the electrical energy delivery circuit, the processor circuit configured to:
identify, in the absence of a diagnosed tachyarrhythmia episode, a first premature ventricular contraction (PVC) of the heart using the received signal representative of electrical activity of the heart and; and
in response to the identified first PVC, initiate delivery of the electrostimulation to be delivered to the location at or near the His bundle using the electrical energy delivery circuit.

2. The apparatus of claim 1, wherein the processor circuit is configured to:
identify, in the absence of a diagnosed tachyarrhythmia episode, an onset of the first PVC; and
in response to the identified onset of the first PVC, initiate delivery of the electrostimulation to be delivered to the location at or near the His bundle during the first PVC.

3. The apparatus of claim 1, wherein the processor circuit is configured to:
identify, in the absence of a diagnosed tachyarrhythmia episode, the first PVC;
identify a non-premature ventricular contraction of the heart, the non-premature ventricular contraction adjacently sequentially precedes the first PVC; and
in response to the identified first PVC, initiate delivery of the electrostimulation to be delivered to the location at or near the His bundle before initiation of a refractory period resulting from the identified non-premature ventricular contraction of the heart.

4. The apparatus of claim 1, wherein the processor circuit is configured to determine a His bundle capture status in response to an electrostimulation delivered to a location at or near the His bundle.

5. The apparatus of claim 4, wherein the processor circuit is configured to adjust an electrostimulation parameter in response to the determined His bundle capture status.

6. The apparatus of claim 5, wherein the processor circuit is configured to adjust at least one of an electrostimulation delay interval or how many electrostimulations are delivered in response to the determined His bundle capture status.

7. The apparatus of claim 1, wherein the processor circuit is configured to:
determine an indication of a first origin of the first PVC of the heart; and
in response to the identified first PVC, initiate delivery of the electrostimulation using the electrical energy delivery circuit and an origin-dependent electrostimulation parameter.

8. The apparatus of claim 1, wherein the electrical energy delivery circuit is configured to provide the electrostimulation to a location in a right ventricle of the heart and on a septum at or near the His bundle.

9. The apparatus of claim 1, wherein the processor circuit is configured to determine a cardiac stimulation indication in response to a delivery of the electrostimulation to the location at or near the His bundle.

10. The apparatus of claim 1, wherein the processor circuit is configured to:
identify, in the absence of a diagnosed tachyarrhythmia episode, the first PVC of the heart using the received signal;
in response to the identified first PVC, initiate delivery of a first electrostimulation to be delivered to the location at or near the His bundle using the electrical energy delivery circuit and an electrostimulation parameter;
identify, in the absence of a diagnosed tachyarrhythmia episode and within a specified first time interval of the first PVC, a subsequent second PVC of the heart using the received signal;
adjust the electrostimulation parameter; and
in response to the identified second PVC, initiate delivery of a second electrostimulation to be delivered to the location at or near the His bundle using the electrical energy delivery circuit and the adjusted electrostimulation parameter.

11. The apparatus of claim 10, wherein the processor circuit is configured to trend information about the first and second electrostimulations.

12. The apparatus of claim 10, wherein the processor circuit is configured to determine a His bundle capture status in response to an electrostimulation delivered to a location at or near the His bundle.

13. The apparatus of claim 12, wherein the processor circuit is configured to determine the His bundle capture status in response to the first electrostimulation; and
when the His bundle capture status indicates non-capture, initiate delivery of the second electrostimulation to the location at or near the His bundle.

14. The apparatus of claim 10, wherein the processor circuit is configured to determine a cardiac stimulation indication in response to at least one of the first electrostimulation or the second electrostimulation.

15. The apparatus of claim 10, wherein the processor circuit is configured to:
in response to the identified first PVC, initiate delivery of the first electrostimulation to be delivered to the location at or near the His bundle, wherein the first electrostimulation includes a first waveform shape, a first waveform amplitude, and a first waveform polarity; and
in response to the identified second PVC, initiate delivery of the second electrostimulation to be delivered to the location at or near the His bundle, wherein the second electrostimulation includes at least one of a different second waveform shape, a different second waveform amplitude, or a different second waveform polarity.

16. The apparatus of claim 10, wherein the processor circuit is configured to trend information about the first electrostimulation, the second electrostimulation, and at least one of a cardiac capture status or a His bundle capture status.

17. A method comprising:
detecting, using a medical device, a first premature ventricular contraction (PVC) of a heart in the absence of a diagnosed tachyarrhythmia episode; and
in response to the detecting the first PVC, using the medical device, automatically delivering a first electrostimulation to a location at or near a His bundle.

18. The method of claim 17, wherein the delivering the first electrostimulation to the location at or near the His bundle includes delivering an electrostimulation to a location at or near at least one of a right ventricle of the heart or a left ventricle of the heart.

19. The method of claim 17, further comprising:
detecting, using the medical device, a subsequent second PVC of the heart in the absence of a diagnosed tachyarrhythmia episode within a specified first time interval of detecting the first PVC;
in response to the detecting the second PVC, adjusting an electrostimulation parameter of the medical device; and
delivering, using the medical device, a second electrostimulation to the location at or near the His bundle using the adjusted electrostimulation parameter;
wherein the adjusting the electrostimulation parameter includes adjusting at least one of an electrostimulation delay interval or how many electrostimulations are delivered.

20. A method comprising:
detecting, using a medical device, a first premature ventricular contraction (PVC) of a heart in the absence of a diagnosed tachyarrhythmia episode;
detecting, using the medical device, an indication of a first origin of the first PVC of the heart;
delivering, using the medical device, a first electrostimulation to a location at or near a His bundle and in response to the detecting the first PVC;
detecting, using the medical device, a subsequent second PVC of the heart in the absence of a diagnosed tachyarrhythmia episode and within a specified first time interval of detecting the first PVC of the heart;
detecting, using the medical device, an indication of a second origin of the second PVC of the heart;
comparing the indication of the detected first origin and the indication of the detected second origin, and:
when the indication of the detected second origin is about the same as indication of the first detected origin, delivering, using the medical device, a second electrostimulation to the location at or near the His bundle using a first electrostimulation parameter of the medical device; and
when the indication of the detected second origin differs from the indication of the detected first origin by greater than a threshold amount, delivering, using the medical device, the second electrostimulation to the location at or near the His bundle using a second electrostimulation parameter of the medical device.
